(19) United States
(12) Patent Application Publication
(10) Pub. No.: US 2017/0340805 A1
(43) Pub. Date: Nov. 30, 2017

(54) Enema Adapter

(71) Applicant: C1R Inc, Los Angeles, CA (US)

(72) Inventor: Robert Novinger, Los Angeles, CA (US)

(21) Appl. No.: 15/605,780

(22) Filed: May 25, 2017

Related U.S. Application Data
(60) Provisional application No. 62/341,518, filed on May 25, 2016.

(51) Int. Cl. A61M 3/02; A61M 39/10

(52) U.S. Cl. CPC A61M 3/0279; A61M 39/10; A61M 3/0262; A61M 3/025; A61M 2039/1033; A61M 2039/1077

(57) ABSTRACT

An enema adapter attaches to a plurality of bottles, shower hoses, or water hoses as the fluid storage vessel or fluid source for body cavity flushing applications, such as enema, douching, or similar applications. The enema adapter directs the flow of water from the into the body cavity for medical or hygienic needs. The enema adapter includes discharge nozzle, a first mounting adapter, at least one discharge opening, and a nozzle cap. The discharge nozzle focuses and directs fluid flow from a fluid reservoir out through the at least one discharge opening into the user's body cavity. The discharge nozzle attaches to the first mounting adapter. The first mounting adapter secures the enema adapter to the fluid reservoir.

The current application claims a priority to the U.S. Provisional Patent application Ser. No. 62/341,518 filed on May 25, 2016.

### FIELD OF THE INVENTION

The present invention relates generally to an adapter for enema applications. More specifically, the present invention is an adapter which can be attached to any water bottle or any shower/water hose with an enema wand component for enema applications.

### BACKGROUND OF THE INVENTION

An enema is a medical procedure involving a fluid being injected into the lower bowel via a rectum, which cleanses the bowel to relieve constipation or for a general cleansing of the bowel before a medical examination or procedure. Similar inventions are also commonly used for vaginal irrigation to rinse the cavity for medical or hygienic reasons. A common method of executing this procedure is through specific bottles with an applicator nozzle. One detriment to this method is that the user is not able to use the applicator nozzle with any bottle and must be used with the specific bottle which may or may not be reusable.

Therefore, it is an objective of the present invention to provide an enema adapter that can be attached to any water bottles or any shower/water hose with an enema wand component for rectal or vaginal use. The present invention provides a first mounting adapter with threading that allows the present invention to be used with any water bottle. The present invention also provides a second mounting adapter with threading that allows the present invention to attach to any shower/water hose. Therefore, the present invention is not limited to one specific water bottle providing the user many options for a container filled with the enema fluid or fluid source.

### BRIEF DESCRIPTION OF THE DRAWINGS

FIG. 1 is an exploded view of the present invention with a water bottle as a fluid reservoir.

FIG. 2 is an exploded view of the present invention with a shower/water hose as fluid reservoir.

FIG. 3 is a perspective view of the discharge nozzle of the present invention.

FIG. 4 is a front view of the discharge nozzle of the present invention.

FIG. 5 is a top view of the discharge nozzle of the present invention.

FIG. 6 is a cross sectional view of the discharge nozzle of the present invention, along the line A-A of FIG. 4.

FIG. 7 is a perspective view of first mounting adapter of the present invention.

FIG. 8 is a front view of the first mounting adapter of the present invention.

FIG. 9 is a top view of the first mounting adapter of the present invention.

FIG. 10 is a bottom view of the first mounting adapter of the present invention.

FIG. 11 is a cross sectional view of the first mounting adapter of the present invention, along the line B-B of FIG. 8.

FIG. 12 is a perspective view of the nozzle cap of the present invention.

FIG. 13 is a perspective view of the second mounting adapter of the present invention.

FIG. 14 is front view of the second mounting adapter of the present invention.

FIG. 15 is a top view of the second mounting adapter of the present invention.

FIG. 16 is a bottom view of the second mounting adapter of the present invention.

FIG. 17 is a front view of the cross-section of first mounting adapter, along the line B-B of FIG. 8, being engaged by second mounting adapter of the present invention.

FIG. 18 is a perspective view of the discharge nozzle and the first mounting adapter of an alternate embodiment for the present invention.

FIG. 19 is a front view of the discharge nozzle and the first mounting adapter of an alternate embodiment for the present invention.

FIG. 20 is a cross-sectional view of the discharge nozzle and the first mounting adapter of an alternate embodiment for the present invention, along the line C-C of FIG. 19.

### DETAIL DESCRIPTIONS OF THE INVENTION

All illustrations of the drawings are for the purpose of describing selected versions of the present invention and are not intended to limit the scope of the present invention.

The present invention is an enema adapter. The present invention is portable and preferably modular such that the user is able to travel with the present invention and clean the present invention effectively. The design of the present invention allows a user to attach the present invention of a plurality of bottles, shower hoses, or water hoses as the fluid storage vessel or fluid source for body cavity flushing applications, such as enema, douching, or similar applications. The present invention directs the flow of water from the into the body cavity for medical or hygienic needs.

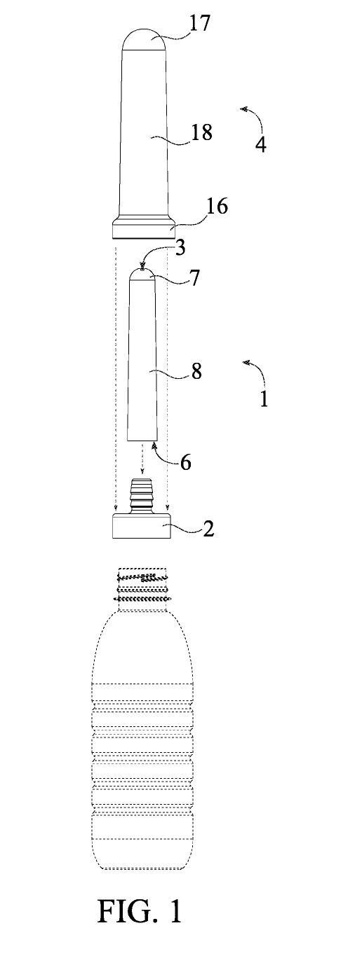

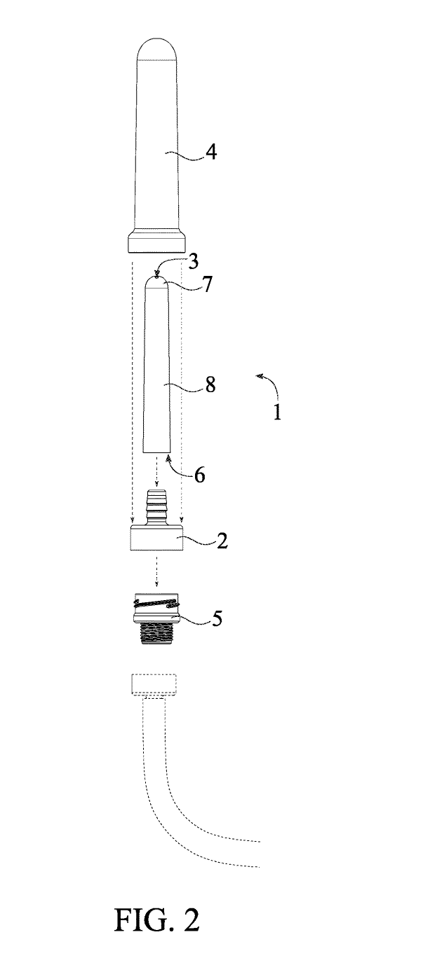

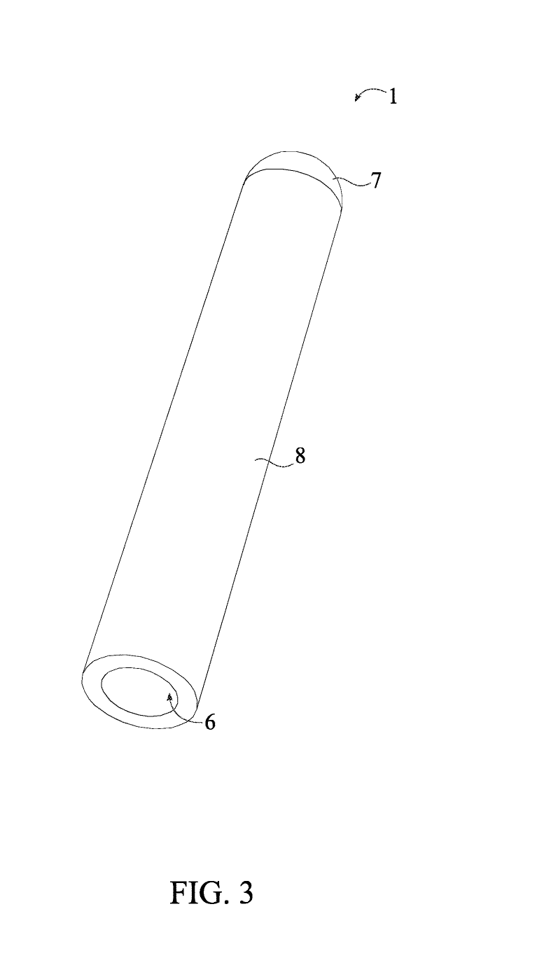

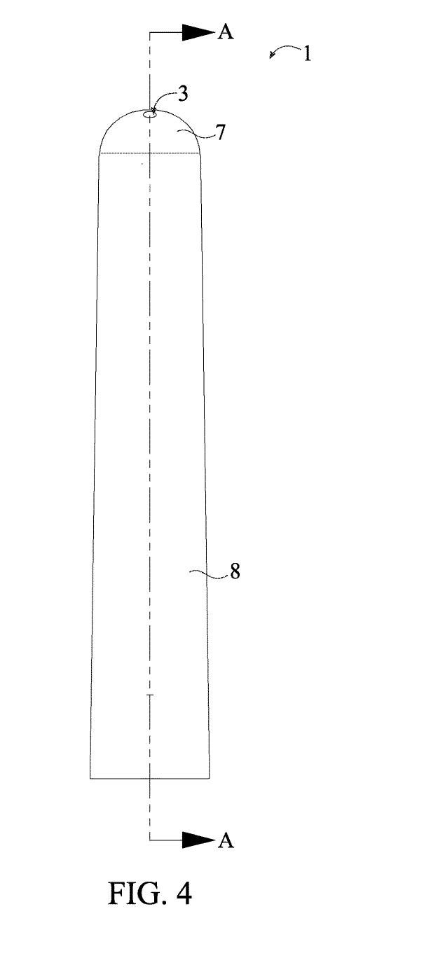

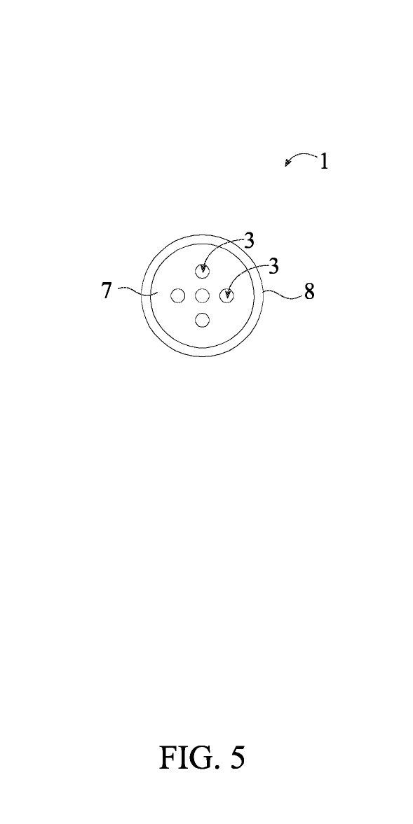

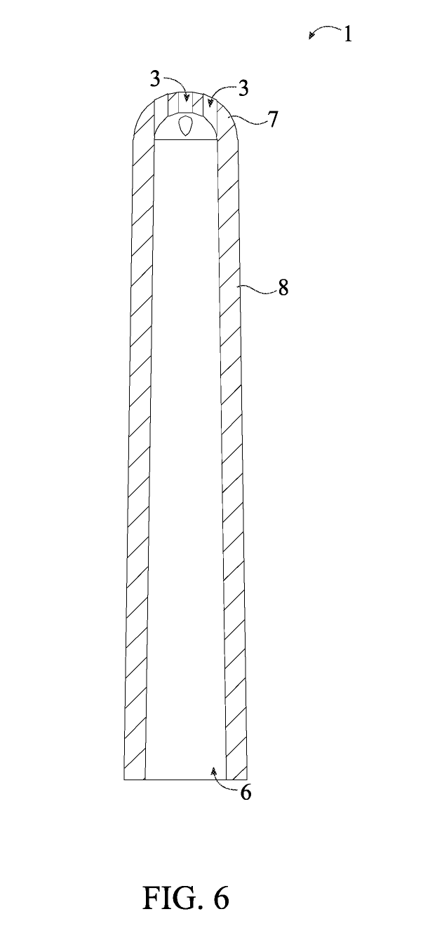

In accordance to FIG. 1 and FIG. 2, the present invention comprises a discharge nozzle 1, a first mounting adapter 2, at least one discharge opening 3, and a nozzle cap 4. The discharge nozzle 1 directs the flow of fluid into the body cavity in order to rinse the body cavity. The discharge nozzle 1 comprises a fluid-receiving opening 6, a fluid-emitting end 7, and a fluid channel 8, as shown in FIG. 1, FIG. 2, FIG. 3 and FIG. 6. The fluid-receiving opening 6 receives fluid from the first mounting adapter 2 to be ejected through the at least one discharge opening 3. The fluid emitting end 7 supports at least one discharge opening 3. fluid emitting end 7 is preferred to be rounded in order to allow for the safe insertion of the discharge nozzle 1 into the appropriate body cavity. The fluid channel 8 allows the transport of the fluid between the fluid-receiving opening 6 and the fluid-emitting end 7. The fluid-receiving opening 6 is oppositely positioned to the fluid-emitting end 7 along the fluid channel 8. The discharge nozzle 1 is adjacently connected to the first mounting adapter 2. The first mounting adapter 2 allows the present invention to be attached to a water bottle or similarly threaded fluid reservoir. The fluid-receiving opening 6 is positioned adjacent to the first mounting adapter 2, in order to receive fluid from the first mounting adapter 2. The at least one fluid discharge opening 3 allows for fluid to egress from the discharge nozzle 1 to flush the desired body cavity, detailed in FIG. 4, FIG. 5, and FIG. 6. The at least one fluid discharge opening 3 traverses through the fluid-emitting end 7, such that the fluid is able to be transported from the water source, through the first mounting adapter 2, into the discharge nozzle 1 and out through the at least one discharge opening 3. The nozzle cap 4 is used to keep the discharge nozzle 1 clean during transportation of the present invention. The nozzle cap 4 is removable attached to the first mounting adapter 2. The discharge nozzle 1 is positioned within the nozzle cap 4 to protect the discharge nozzle 1 from dust, debris, bacteria, and damage during transportation.

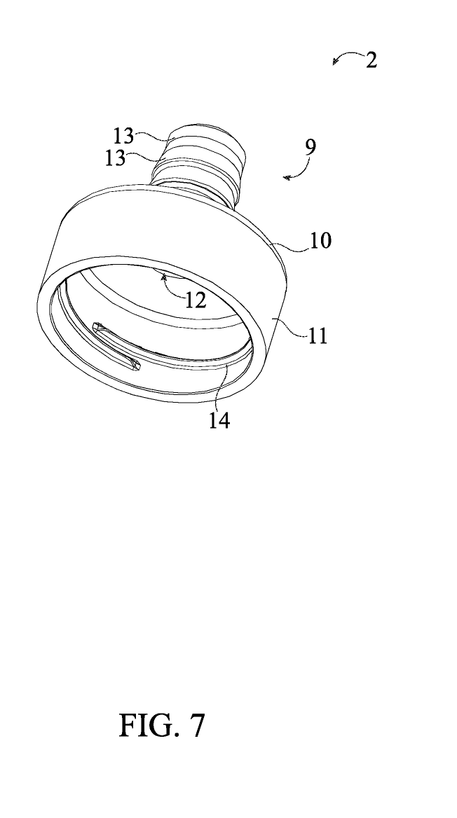

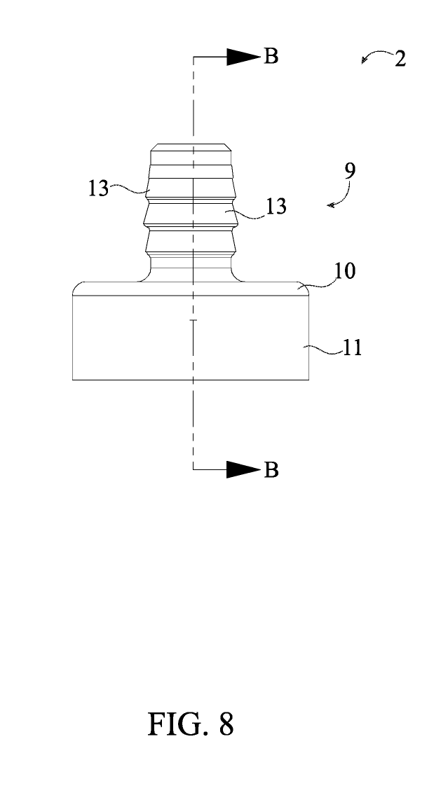

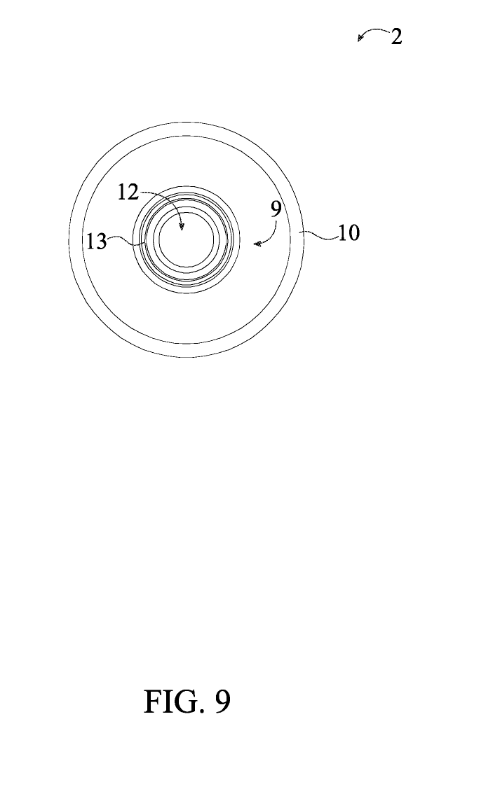

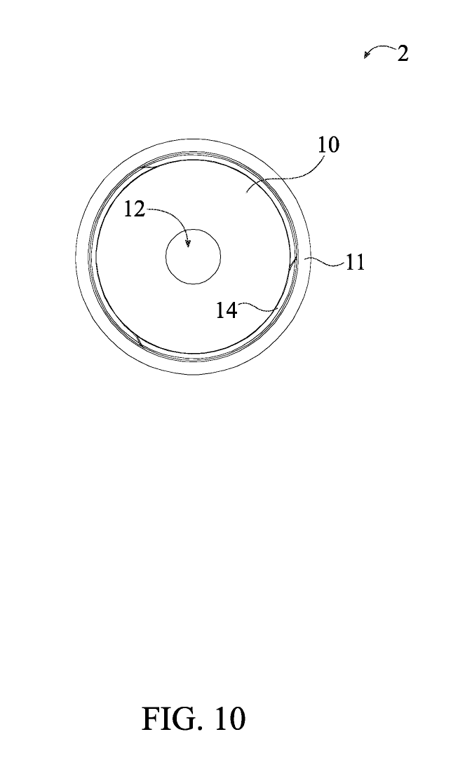

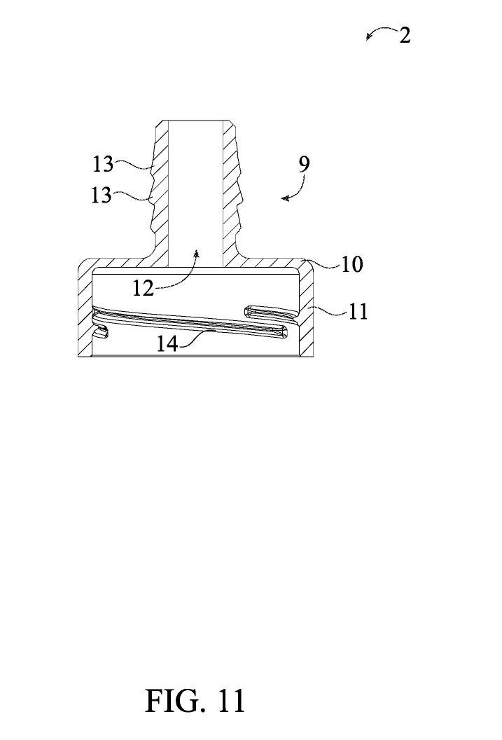

In accordance to the preferred embodiment of the present invention, the first mounting adapter 2 comprises a nozzle-attachment extrusion 9, a first adapter plate 10, a primary mounting extrusion 11, and a first fluid transport opening 12, as detailed in FIG. 7. The nozzle-attachment extrusion 9 allows for the discharge nozzle 1 to be secured to the first mounting adapter 2 easily. The nozzle-attachment extrusion 9 is adjacently connected to the first adapter plate 10, as detailed in FIG. 8. The first adapter plate 10 supports the nozzle-attachment extrusion 9, the primary mounting extrusion 11 and the first fluid transport opening 12. The primary mounting extrusion 11 secures the first mounting adapter 2 to the fluid reservoir. The primary mounting extrusion 11 is perimetrically connected to the first adapter plate 10, as shown in FIG. 10. The primary mounting extrusion 11 is oppositely positioned to the nozzle-attachment extrusion 9 about the first adapter plate 10, in accordance to FIG. 7, FIG. 8 and FIG. 11. The first fluid transport opening 12 allows fluid to be transferred through the first mounting adapter 2 to the discharge nozzle 1. The first fluid transport opening 12 centrally traverses through the first adapter plate 10, detailed in FIG. 10 and FIG. 11. More specifically, the nozzle-attachment extrusion 9 is perimetrically positioned around the fluid transport opening, shown in FIG. 9, in order to reduce head loss through the first mounting adapter 2. The nozzle-attachment extrusion 9 is oriented normal to the first adapter plate 10, in order to further reduce head loss through the first mounting adapter 2.

In accordance to more specific embodiment of the present invention, the present invention comprises a plurality of sloping ridges 13, shown in FIG. 7, FIG. 8, and FIG. 11. The plurality of sloping ridges 13 increase frictional forces between the nozzle-attachment extrusion 9 and the discharge nozzle 1. The plurality of sloping ridges 13 is integrated around the nozzle-attachment extrusion 9. Each of the plurality of sloping ridges 13 is offset from one another along the nozzle-attachment extrusion 9, in order for sloping ridges of plurality of sloping ridges 13 to engage the discharge nozzle 1 as the discharge nozzle 1 is attached to the nozzle-attachment extrusion 9.

Further in accordance to the preferred embodiment of the present invention, the present invention comprises a first interlocking-adapter threading 14, detailed in FIG. 7, FIG. 10 and FIG. 11. The first interlocking-adapter threading 14 allows the first mounting adapter 2 to be secured to a water bottle or similarly threaded fluid reservoirs. The first interlocking-adapter threading 14 is internally connected about the primary mounting extrusion 11, in order to receive the threading of the water bottle or similarly threaded fluid reservoir.

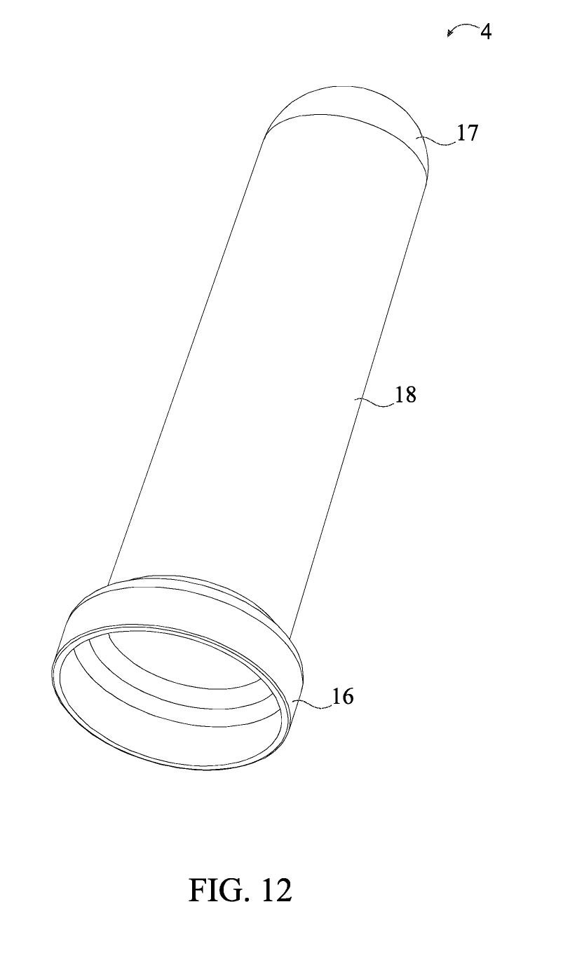

Still in accordance to the preferred embodiment of the present invention, the nozzle cap 4 comprises an adapter-receiving end 16, a closed-cap end 17, and an enclosure tube 18, shown in FIG. 12. The adapter-receiving end 16 secures the nozzle cap 4 to the first mounting adapter 2 through a snap connection. The closed-cap end 17 and the enclosure tube 18 encapsulate the discharge nozzle 1 in order to protect the discharge nozzle 1 from dust, debris, and damage during transfer of the present invention. The adapter-receiving end 16 being perimetrically connected to the enclosure tube 18. The closed-cap end 17 being perimetrically connected to the enclosure tube 18. The adapter-receiving end 16 being oppositely positioned to the closed-cap end 17 along the enclosure tube 18. The adapter-receiving end 16 engaging the first mounting adapter 2, in order to encapsulate the discharge nozzle 1.

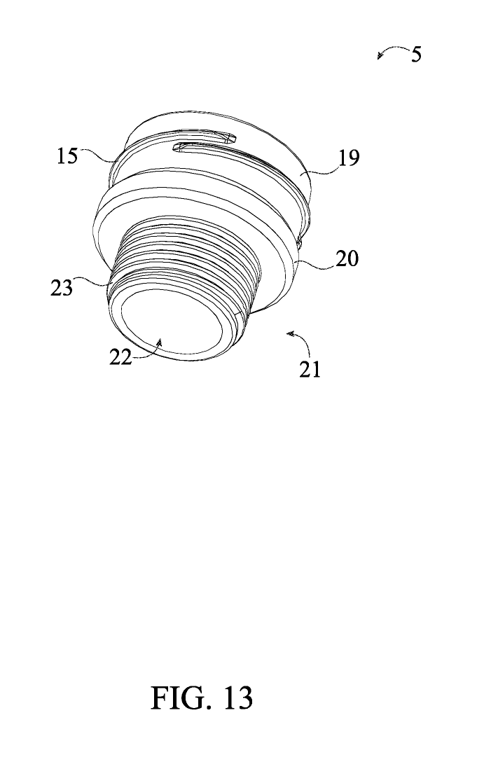

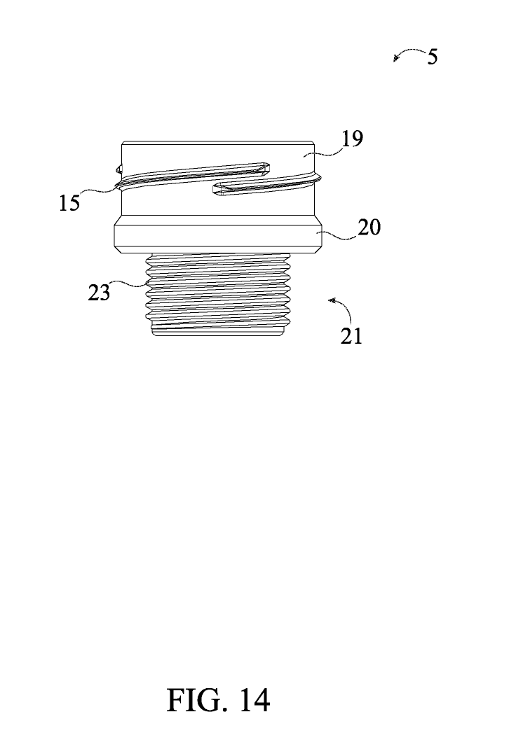

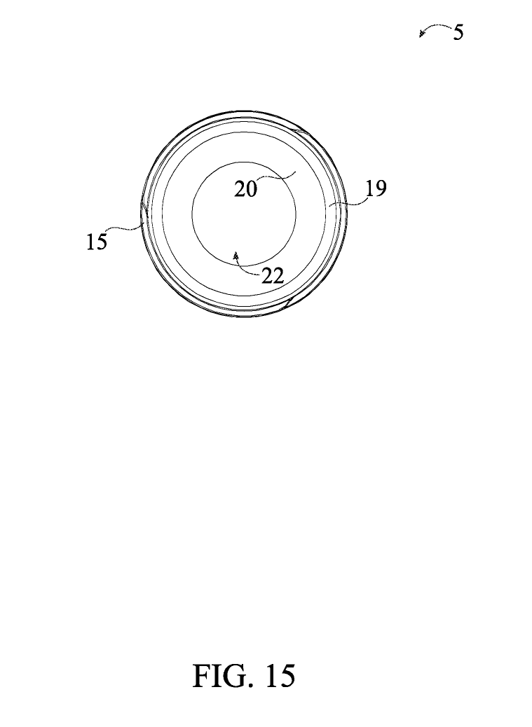

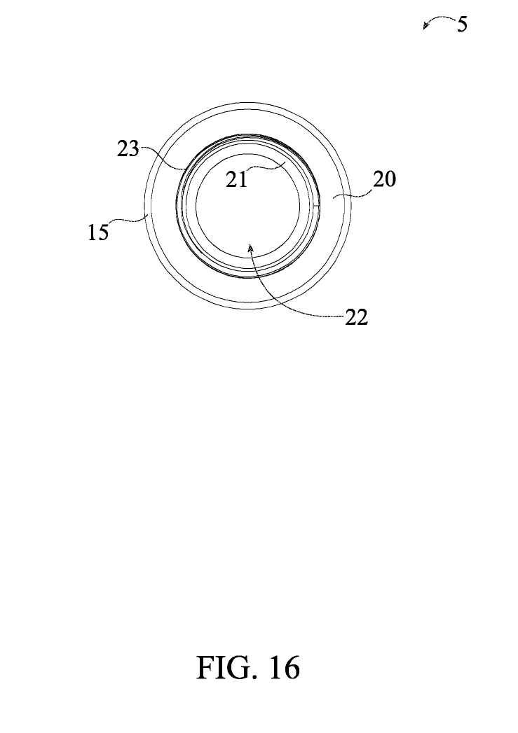

In some embodiments of the present invention, the present invention comprises a second mounting adapter 5. The second mounting adapter 5 comprises a first adapter-mounting extrusion 19, a second adapter plate 20, a secondary mounting extrusion 21, and a second fluid transport opening 22, in accordance to FIG. 13. The first adapter-mounting extrusion 19 secures the second mounting adapter 5 to the first mounting adapter 2. The first adapter-mounting extrusion 19 is perimetrically connected to the second adapter plate 20, in accordance to FIG. 15. The second adapter plate 20 supports the first adapter-mounting extrusion 19, the secondary mounting extrusion 21, and the second fluid transport opening 22. The secondary mounting extrusion 21 attaches the second mounting adapter 5 to a water hose, a shower hose, or similarly threaded fluid source plumbing. The secondary mounting extrusion 21 is adjacently and centrally connected to the second adapter plate 20, detailed in FIG. 16. The secondary mounting extrusion 21 is positioned opposite to the first adapter-mounting extrusion 19 about the second adapter plate 20, shown in FIG. 14. The first adapter-mounting extrusion 19 engages the first mounting adapter 2. The second fluid transport opening 22 allows fluid to be transferred from the threaded fluid-source plumbing through the second mounting adapter 5 and into the first mounting adapter 2, in accordance to FIG. 13, FIG. 15, and FIG. 16. The second fluid transport opening 22 centrally traverses through the second adapter plate 20. More specifically, the secondary mounting extrusion 21 is perimetrically positioned around the second fluid transport opening 22, in order to reduce head loss through the second mounting adapter 5.

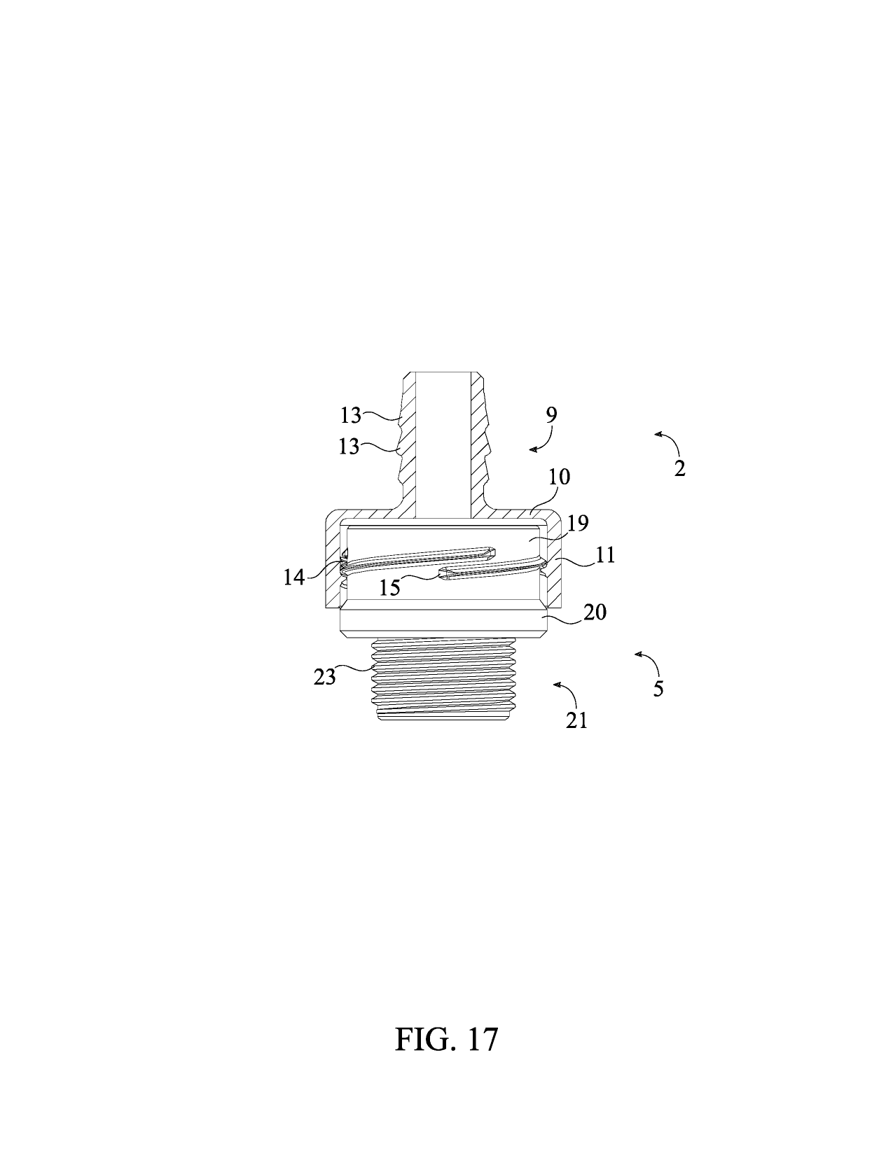

Further in accordance to these embodiments of the present invention, the present invention comprises a second interlocking-adapter threading 15 and a mounting threading 23, detailed in FIG. 13, FIG. 14, and FIG. 17. The second interlocking-adapter threading 15 secures the second mounting adapter 5 to the first mounting adapter 2. The second interlocking-adapter threading 15 being externally connected around the first adapter-mounting extrusion 19, shown in FIG. 13, FIG. 14, FIG. 15, and FIG. 17. The first adapter-mounting extrusion 19 engaging the first mounting adapter 2 by the second interlocking-adapter threading 15, in order to secure the second mounting adapter 5 to the first mounting adapter 2. The mounting threading 23 secures the second mounting adapter 5 to the threaded fluid-source plumbing. The mounting threading 23 being externally connected around the secondary mounting extrusion 21, shown in FIG. 13, FIG. 14, FIG. 16, and FIG. 17, in order to attach the second mounting adapter 5 to a shower hose, water hose, or similarly threaded fluid-source plumbing.

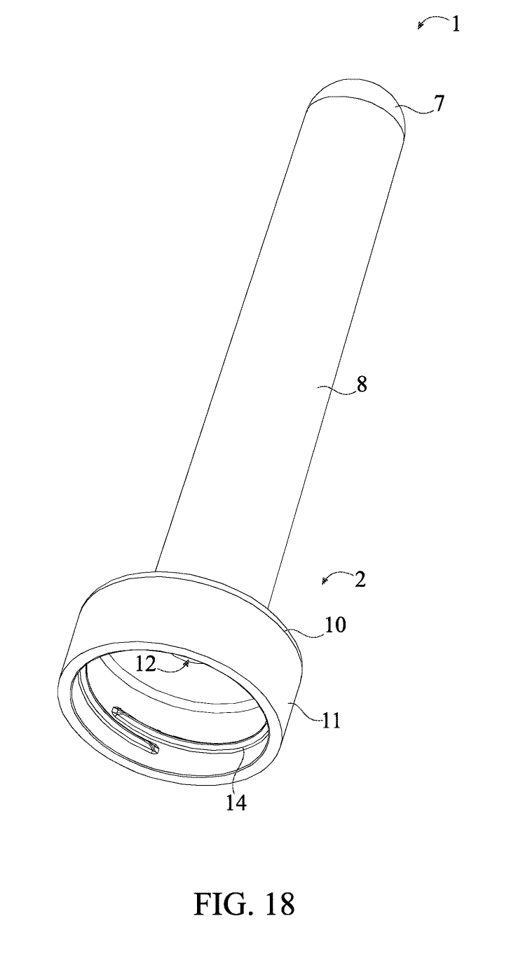

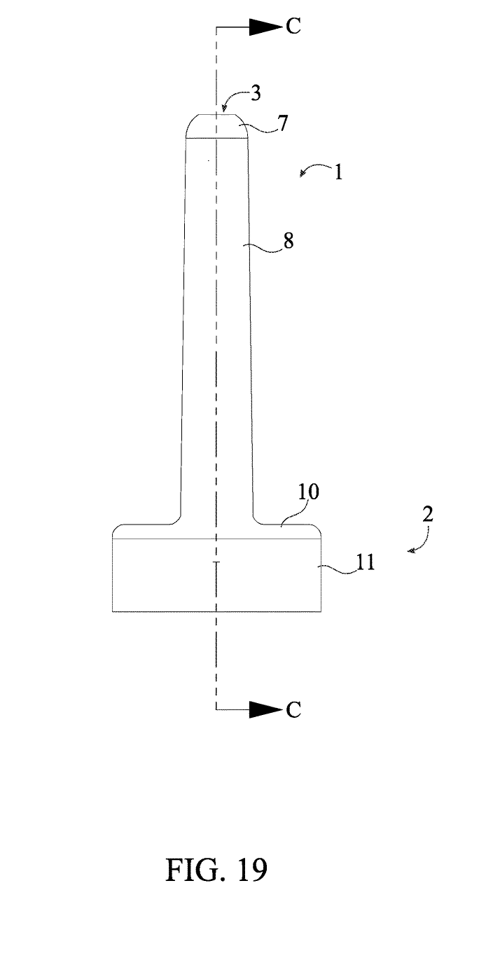

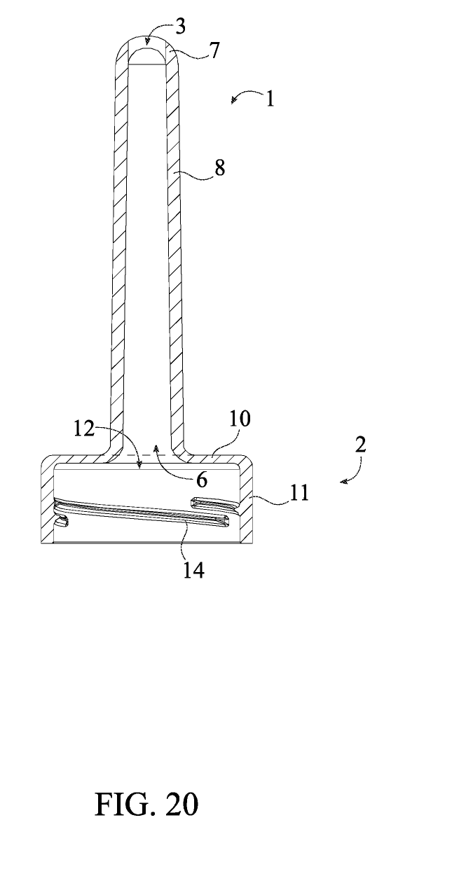

In accordance to an alternate embodiment of the present invention, the first mounting adapter comprises a first adapter plate 10, a primary mounting extrusion 11, and a first fluid transport opening 12, shown in FIG. 18. Similar to the preferred embodiment of the present invention, the first adapter plate 10 supports the discharge nozzle 1, the primary mounting extrusion 11 and the first fluid transport opening 12 The discharge nozzle 1 is adjacently connected to the first adapter plate 10, as shown in FIG. 18, FIG. 19, and FIG. 20. The primary mounting extrusion 11 secures the first mounting adapter 2 to the fluid reservoir. The primary mounting extrusion 11 is perimetrically connected to the first adapter plate 10. The primary mounting extrusion 11 is oppositely positioned to the discharge nozzle 1 about the first adapter plate 10, detailed in FIG. 18, FIG. 19, and FIG. 20. The first fluid transport opening 12 allows fluid to be transferred through the first mounting adapter 2 to the discharge nozzle 1. The first fluid transport opening 12 centrally traverses through the first adapter plate 10, in accordance to FIG. 18 and FIG. 20. More specifically, the discharge nozzle 1 is perimetrically positioned around the first fluid transport opening 12, in order to reduce head loss through the first mounting adapter 2. The discharge nozzle 1 is oriented normal to the first adapter plate 10, in order to further reduce head loss through the first mounting adapter 2.

Although the invention has been explained in relation to its preferred embodiment, it is to be understood that many other possible modifications and variations can be made without departing from the spirit and scope of the invention as hereinafter claimed.

What is claimed is:

1. An enema adapter comprises:
a discharge nozzle;
a first mounting adapter;
at least one fluid discharge opening;
a nozzle cap;
the discharge nozzle comprises a fluid-receiving opening, a fluid-emitting end, and a fluid channel;
the fluid-receiving opening being positioned opposite to the fluid-emitting end along the fluid channel;
the discharge nozzle being adjacently connected to the first mounting adapter;
the fluid-receiving opening being positioned adjacent to the first mounting adapter;
the at least one fluid discharge opening traversing through the fluid-emitting end;
the nozzle cap being removably attached to the first mounting adapter; and
the discharge nozzle being positioned within the nozzle cap.

2. The enema adapter, as claimed in claim 1, comprises:
the first mounting adapter comprises a nozzle-attachment extrusion, a first adapter plate, a primary mounting extrusion, and a first fluid transport opening;
the nozzle-attachment extrusion being adjacently connected to the first adapter plate;
the primary mounting extrusion being perimetrically connected to the first adapter plate;
the primary mounting extrusion being oppositely positioned to the nozzle-attachment extrusion about the first adapter plate; and
the first fluid transport opening centrally traversing through the first adapter plate.

3. The enema adapter, as claimed in claim 2, comprises:
the nozzle-attachment extrusion being perimetrically positioned around the first fluid transport opening.

4. The enema adapter, as claimed in claim 2, comprises:
the nozzle-attachment extrusion being oriented normal to the first adapter plate.

5. The enema adapter, as claimed in claim 2, comprises:
a plurality of sloping ridges;
the plurality of sloping ridges being integrated around the nozzle-attachment extrusion; and
each of the plurality of sloping ridges being offset from one another along the nozzle-attachment extrusion.

6. The enema adapter, as claimed in claim 2, comprises:
a first interlocking-adapter threading; and
the first interlocking-adapter threading being internally connected about the primary mounting extrusion.

7. The enema adapter, as claimed in claim 1, comprises:
the nozzle cap comprises an adapter-receiving end, a closed-cap end, and an enclosure tube;
the adapter-receiving end being perimetrically connected to the enclosure tube;
the closed-cap end being perimetrically connected to the enclosure tube;
the adapter-receiving end being oppositely positioned to the closed-cap end along the enclosure tube; and
the adapter-receiving end engaging the first mounting adapter.

8. The enema adapter, as claimed in claim 1, comprises:
a second mounting adapter;
the second mounting adapter comprises a first adapter-mounting extrusion, a second adapter plate, a secondary mounting extrusion, and a second fluid transport opening;
the first adapter-mounting extrusion being perimetrically connected to the second adapter plate;
the secondary mounting extrusion being adjacently and centrally connected to the second adapter plate;
the secondary mounting extrusion being positioned opposite to the first adapter-mounting extrusion about the second adapter plate;
the first adapter-mounting extrusion engaging the first mounting adapter; and
the second fluid transport opening centrally traversing through the second adapter plate.

9. The enema adapter, as claimed in claim 8, comprises:
the secondary mounting extrusion being perimetrically positioned around the second fluid transport opening.

10. The enema adapter, as claimed in claim 8, comprises:
a second interlocking-adapter threading;
the second interlocking-adapter threading being externally connected around the first adapter-mounting extrusion; and
the first adapter-mounting extrusion engaging the first mounting adapter by the second interlocking-adapter threading.

11. The enema adapter, as claimed in claim 8, comprises:
a mounting threading; and
the mounting threading being externally connected around the secondary mounting extrusion.

12. The enema adapter, as claimed in claim 1, comprises:
the first mounting adapter comprises a first adapter plate, a primary mounting extrusion, and a first fluid transport opening;
the discharge nozzle being adjacently connected to the first adapter plate;
the primary mounting extrusion being perimetrically connected to the first adapter plate;
the primary mounting extrusion being oppositely positioned to the discharge nozzle about the first adapter plate; and
the first fluid transport opening centrally traversing through the first adapter plate.

13. The enema adapter, as claimed in claim 12, comprises:
the discharge nozzle being perimetrically positioned around the first fluid transport opening.

14. The enema adapter, as claimed in claim 12, comprises:
the discharge nozzle being oriented normal to the first adapter plate.
